(19) United States
(12) Patent Application Publication
(10) Pub. No.: US 2014/0080161 A1
(43) Pub. Date: Mar. 20, 2014

(54) PHOSPHATASE COUPLED KINASE ASSAY

(71) Applicant: Zhenliang L. Wu, Edina, MN (US)

(72) Inventor: Zhenliang L. Wu, Edina, MN (US)

(73) Assignee: RESEARCH AND DIAGNOSTIC SYSTEMS, INC., Minneapolis, MN (US)

(21) Appl. No.: 13/822,056

(22) PCT Filed: Sep. 14, 2010
(86) PCT No.: PCT/US10/48747

§ 371 Date: Mar. 11, 2013

(51) Int. Cl. C12Q 1/48

(52) U.S. Cl. CPC C12Q 1/485
USPC 435/15

(57) ABSTRACT

Assays and methods for detecting and quantifying the activity of a test kinase. The assay includes a nucleotide phosphatase at least ten times more active on ADP than on ATP and a free phosphate detector. The assay may also include a control kinase and/or ATP. Methods of the invention include combining a test kinase, a substrate of the test kinase, and a known quantity of ATP under conditions to produce ADP, combining the produced ADP with a phosphatase specific for ADP, and measuring free phosphate. The amount of phosphate produced directly correlates to the activity of the kinase.phosphatase coupled kinase assay

### BACKGROUND

Kinases act to phosphorylate a large variety of substrates, and this phosphorylation plays a role in nearly every cellular activity. For example kinases are key to biological processes such as intracellular signal transduction and enzymatic regulation. Through phosphorylation by kinases, extracellular signals can be relayed to the cell nucleus which can in turn signal the cell to various activities such as growth, migration or rest. Because of the key role of kinase activity in cellular functioning, it is desirable to measure kinase activity. However, measurement of kinase activity has been difficult. Various methods exist, but each has certain disadvantages.

One method of measuring kinase activity detects ADP produced by a kinase using a series of reactions, with the final reaction being measurable. ADP is first coupled to the production of pyruvate through pyruvate kinase, and the pyruvate is then oxidized by pyruvate oxidase to produce hydrogen peroxide. The hydrogen peroxide is then utilized by peroxidase to generate resorufin, which has a detectable fluorescence. This multistep process generates a signal which is removed from the original kinase reaction by several steps. In addition to being cumbersome, the multistep nature of this test results in decreased accuracy, with the final signal being a less accurate measurement of the kinase reaction than a system having fewer steps would be.

Another method of measuring kinase activity converts the ADP produced by the kinase back to ATP, which is then detected by a lucifierase. This type of assay also requires on multiple steps, which results in decreased accuracy. In addition, for this assay to work properly, the remaining reactant ATP must be depleted without changing the level of product ADP, and the product ADP must be completely converted back to ATP through another coupled enzymatic reaction. These processes are complicated and difficult to achieve.

Another method of measuring kinase activity uses an ADP specific monoclonal antibody which may need to be conjugated to a quencher or a donor label as well as a fluorescein-conjugates ADP tracer. The production of the conjugated antibody and conjugated ADP, with the conjugated groups being joined in the correct locations, are complicated and delicate processes. Also, because the method is based on competitive binding of ADP and the ADP-conjugated tracer to the antibody, the produced signal is not linearly related to the product ADP. As such, the production of this assay, as well as the use of this assay, are both complicated.

### SUMMARY

Embodiments of the invention include assay, kits, and methods for detecting and quantifying kinase activity. Kinase activity results in the production of ADP, and embodiments of the invention use an ADP specific phosphatase to release free phosphate from the ADP and then detect the free phosphate. The resulting free phosphate directly correlates to the production of ADP and therefore to the kinase activity.

In some embodiments, an assay for detecting activity of a test kinase includes a nucleotide phosphatase and a free phosphate detector. The phosphatase is at least about ten times more active on ADP than on ATP. In some embodiments, the assay also includes a control kinase, such as Saccharomyces cerevisiae hexokinase. In other embodiments, the assay also includes ATP. In still other embodiments, the assay also includes both a control kinase and ATP. The assay may also include a source of free phosphate for use as a phosphate standard.

In some embodiments, a method of detecting kinase activity includes conducting a first reaction including combining a kinase, a substrate of the kinase, and a known quantity of ATP under conditions to produce ADP, combining the produced ADP with a phosphatase specific for ADP, and measuring free phosphate. The phosphatase may be combined with the kinase, substrate, and ATP at the beginning of the kinase reaction, with all components combined at the same time, or the phosphatase may be added to the kinase reaction after the kinase reaction has progressed for a period of time.

In some embodiments, the method also includes calculating kinase activity using the measured amount of free phosphate. The method may also include adjusting the measured free phosphate according to the coupling efficiency at the ATP concentration used in the reaction. This allows for accurate kinetic measurement of kinase reactions even at high ATP concentration. For example, the method can be performed using ATP concentrations at or above physiological ATP concentration, such as about 1 mM or greater than about 1 mM.

In some embodiments, the method includes conducting a second reaction to provide a background control. The control reaction may include all of the components of the first reaction except the kinase. For example, in some embodiments, conducting the second reaction or control reaction includes combining the substrate of the kinase and the known quantity of ATP with the phosphatase and measuring free phosphate. The method may further include reducing the measured phosphate of the first reaction by the measured phosphate of the second reaction to calculate the corrected amount of phosphate produced by the kinase.

The phosphatase used in assays and method of the invention may be at least about ten times more active on ADP than on ATP. In some embodiments, the phosphatase is at least about thirty times more active on ADP than on ATP, while in still other embodiments, the phosphatase is at least about seventy times more active on ADP than on ATP. In some embodiments, the phosphatase is an ectonucleoside triphosphate diphosphohydrolase, such as ectonucleoside triphosphate diphosphohydrolase 6.

The free phosphate detector may be a colorimetric assay. For example, in some embodiments, the free phosphate detector comprises a first reagent and a second reagent, wherein the first reagent includes ammonium molybdate and the second reagent includes malachite green oxalate. In some embodiments, measuring the free phosphate includes measuring optical density.

### FIGURES

The following figures are illustrative of particular embodiments of the present invention and therefore do not limit the scope of the invention. The figures are intended for use in conjunction with the explanations in the following detailed description.

FIG. 1 is an exemplary reaction according to embodiments of the invention;

FIG. 2 is a hydrolysis curve for fixed amounts of ADP and ATP using increasing amounts of rhCD39L2;

FIG. 3 is a curve of absorbance versus ADP concentration with a constant amount of CD39L2 for various reaction times;

FIG. 4 is a curve of absorbance versus ADP concentration at various constant concentrations of ATP;

FIG. 5 is a curve of coupling efficiency versus ATP concentration;

FIG. 6 is chart of absorbance for hexokinase reactions including various substrates;

FIG. 7 is a curve of absorbance versus amount of hexokinase;

FIG. 8a is a curve of absorbance versus ATP concentration for hexokinase;

FIG. 8b is a fit curve for the final curve of FIG. 8a;

FIG. 9 is a curve of absorbance versus quantity of APSK;

FIG. 10 is a curve of absorbance versus quantity of ERK1;

FIG. 11 is a hydrolysis curve for ADP and ATP using rmCD39L2, and

FIG. 12 is a hydrolysis curve for ADP and ATP using rhCD39L4.

### DETAILED DESCRIPTION

Embodiments of the invention include assays and methods for detecting and quantitatively measuring kinase activity by measuring the amount of ADP which is released as a byproduct of a kinase activity. Because all kinases consume ATP and produce ADP, the invention has universal application and can be applied to measure the activity of any kinase. Furthermore, because the amount of ADP produced directly correlates to the activity of the kinase, the measurement of ADP provides a quantitative measurement of the kinase activity. Embodiments of the invention can be used not only for characterizing kinase activity but also for assessing the effect of various agents upon kinase activity. As such, they may be used for screening and for analyzing various agents for their effect as promoters or inhibitors of kinase activity.

Embodiments of the invention use a nucleotide phosphatase having preferential activity with ADP as compared to ATP to remove a phosphate from ADP produced by a kinase. The level of free phosphate is then measured to detect and quantify kinase activity. For each phosphate transfer performed by the kinase, one ADP is produced and one phosphate is available to be released by the phosphatase. The amount of phosphate released can then be detected and measured, thereby providing a quantitative measurement of the kinase activity. Embodiments of the invention therefore provide methods of measuring the kinetics of kinase reactions using non-radioactive methods. Furthermore, the reactions can be performed in a multi-well plate and read with a multi-well plate reader, making them useful for high throughput screening.

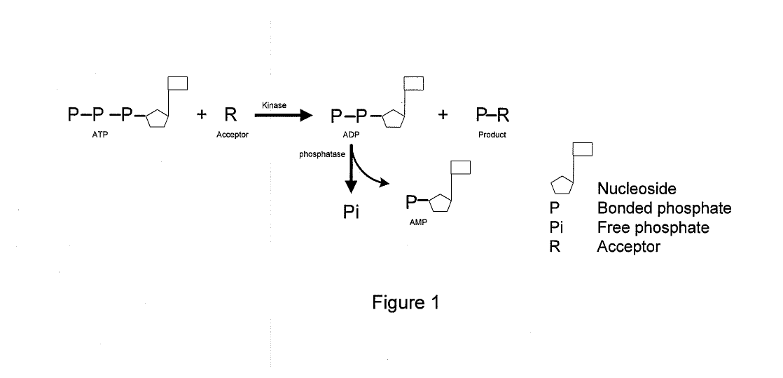

An example of a reaction according to embodiments of the invention is shown in FIG. 1. The reaction begins with an ATP, an acceptor and a kinase. The kinase transfers one phosphate from the ATP to the acceptor, producing one molecule of ADP and a product (which is a phosphorylated acceptor). A nucleotide phosphatase having preferential activity for ADP then removes one phosphate from the ADP, producing one free inorganic phosphate and one molecule of AMP. Because the phosphatase is a nucleotide phosphatase, it only removes phosphate from nucleotides and it does not remove the phosphate from the acceptor. Furthermore, because the phosphatase has preferential activity with ADP versus ATP, it does not remove phosphate from ATP (or removes phosphate from ATP at a much slower rate). The free inorganic phosphate released from the ADP can then be detected and measured, such as by a colorimetric assay. As can be seen, the amount of free phosphate produced directly correlates with the production of ADP and therefore the progress of the kinase reaction. Unlike other kinase assays, embodiments of the invention do not require multiple reaction steps, but rather the produced ADP can be easily measured using a single reaction, without requiring separation of the ADP from the ATP. As a result, the signal measurement is easier and more accurate than existing kinase assays.

Because the reaction may require the phosphatase to act in the presence of both ATP and ADP, embodiments of the invention employ phosphatases having greater activity for ADP than for ATP. However, even if the phosphatase has some activity with ATP, the measurement of free phosphate can be adjusted as described below to account for this activity so that it nevertheless does correlate with kinase activity.

The relative activity of the phosphatase on ADP and ATP can be measured by creating hydrolysis curves for ADP and ATP. For example, such hydrolysis curves may be created by performing a series of reactions separately combining set quantities of ADP or ATP with varying quantities of phosphatase and detecting the free phosphate produced. An example of such curves is described in Example 1 below. The resulting phosphate levels (or OD readings, which directly correspond to the phosphate levels) can be plotted against the amount of phosphatase used. The phosphate level or OD readings can be compared at the same quantity of the phosphatase to determine the relative activity of the phosphatase for ADP as compared to ATP. Such hydrolysis curves may exhibit an S-shaped curve, including a central portion of the curve which is most rapidly rising (steepest). The amount of phosphatase to be used for kinase reactions is preferably selected from within the rapidly rising portion of the ADP hydrolysis curve. In addition, the relative activity of the phosphatase for ADP versus ATP is preferably compared within this rapidly rising portion of the hydrolysis curve, at or near the steepest portion of the curve. At phosphatase levels below this range, the generated signals are weak, while at phosphatase levels above this range, the ADP hydrolysis approaches completion, which could underestimate the activity of the phosphatase on ADP due to exhaustion of the substrate.

In order to be useful in embodiments of the invention, phosphatases should have a higher activity with ADP than with ATP. The relative activity may be compared by using hydrolysis curves as described above. In some embodiments, the phosphatase is at least 10 times more active on ADP than on ATP. In other embodiments, the phosphatase is at least about 30 times more active on ADP than on ATP, while in still other embodiments, the phosphatase is at least about 70 times more active on ADP than on ATP.

Phosphatases used in embodiments of the invention have a high specific activity on ADP. The specific activity of the phosphatase used in the examples is the amount of free phosphate released from a substrate per unit time and per amount of phosphatase under a given set of conditions, and may be determined at the rising phase of an enzymatic dose curve, such as the hydrolysis curve as described above. In some embodiments, the phosphatase has a specific activity of greater than 100 pmol/min/μg under optimal conditions. The conditions may include optimal pH, salt concentrations, and the presence of any cofactor. In other embodiments, the phosphatase has a specific activity of greater than 1,000 pmol/min/μg. In still other embodiments, the phosphatase has a specific activity of greater than 20,000 pmol/min/μg. When phosphatases having lower specific activity are used, larger amounts of such phosphatases or longer reaction times may be used to achieve the same level of hydrolysis as can be achieved with a phosphatase having higher specific activity.

In some embodiments, a kinase reaction is performed by combining a kinase, a substrate of the kinase, ATP and a phosphatase at the same time. As ADP is produced by the kinase, it is hydrolyzed by the phosphatase to release free phosphate. At the end of the reaction or after the reaction is stopped, the free phosphate can then be measured. Alternatively, the kinase reaction and the phosphatase reaction can be performed separately. In such embodiments, a kinase, a substrate of the kinase, and ATP are combined and allowed to react for a certain period of time. The kinase reaction may then optionally be stopped. A phosphatase is then added to the reactants and allowed to react with the produced ADP. The reaction(s) are then stopped and the free phosphate can then be measured. The phosphatase reaction may be initiated at a later stage, after initiation of the kinase reaction under certain situations, such as when the kinase reaction is slow, when the kinase has a much lower activity than the phosphatase, or when the kinase buffer or other component of the kinase assay is not compatible with the phosphatase or would interfere with the phosphatase activity.

One phosphatase which may be used in embodiments of the invention is CD39L4, also known as ectonucleoside triphosphate disphosphohydrolase 5 (NTPDase5). CD39L4 is about 30 to 40 times more active on ADP than on ATP. Furthermore, it has a specific activity of >1,000 pmol/min/μg on ADP. Another phosphatase which may be used in embodiments of the invention is CD39L2, also known as ectonucleoside triphosphate diphosphohydrolase 6 (NTPDase6). CD39L2 is about 70 to 80 times more active on ADP than on ATP and has a specific activity of >20,000 pmol/min/μg on ADP. As such, it is highly active on ADP and is much more active on ADP than on ATP, making it a useful phosphatase for embodiments of the invention.

Phosphatases which may be used in embodiments of the invention may be isolated from naturally occurring sources. Alternatively, they may be produced through recombinant DNA methods. For example, CD39L2 useful in embodiments of the invention may be recombinant human CD39L2 (rhCD39L2) or recombinant mouse CD39L2 (rmCD39L2), or may be recombinantly produced from other animal or mammalian sources. Furthermore, it is expected that, through further research, phosphatases could be modified through recombinant protein engineering, for example, to increase their relative preference for ADP, and embodiments of the invention would also include modified CD39L4 and CD39L2 and derivatives of CD39L4 and CD39L2, as well as homologues from different organisms. Furthermore, any phosphatase having a sufficient preference for ADP over ATP may be used in embodiments of the invention. For example, any phosphatase having a specific activity of greater than about 100 pmol/min/μg on ADP and being at least 10 times more active on ADP than on ATP may be used in embodiments of the invention. Known phosphatases, as well as those isolated or developed in the future, may be screened for preferential activity with ADP as compared to ATP using hydrolysis curves, for example. When such preference is discovered, such phosphatases may also be used in embodiments of the invention. Furthermore, such phosphatases may be further modified to increase their preference for ADP, increasing their usefulness in embodiments of the invention, and such phosphatases are therefore also included in embodiments of the invention.

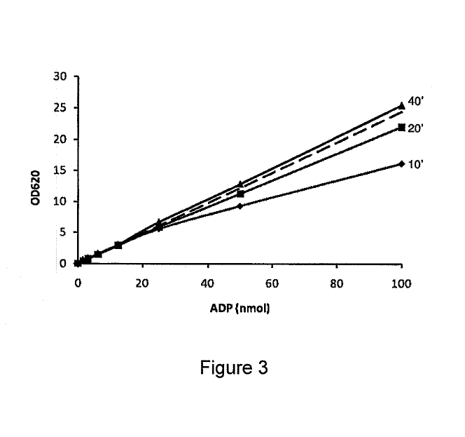

Phosphatases useful in embodiments of the invention are preferably able to completely hydrolyze all of the ADP generated in a kinase reaction. The amount of complete hydrolysis which can be performed by a phosphatase under a set of reaction conditions may be characterized as the coupling capacity of the phosphatase. As such, the coupling capacity represents the amount of ADP that can be completely hydrolyzed by a given amount of a phosphatase under specific conditions. Coupling capacity may be determined by performing a series of reactions, combining a set amount of phosphatase with increasing amounts of ADP. The reactions may be performed for various lengths of time, with different coupling capacities as a result. The desired reaction time and amount of phosphatase to be used in kinase reactions can then be selected such that the corresponding coupling capacity is greater than the anticipated amount of ADP produced in the kinase reaction. An example of an experiment to determine coupling capacity is provided in Example 2 and shown in FIG. 3. Coupling capacity can be determined as the maximum amount of ADP that is completely hydrolyzed by a given amount of phosphatase in FIG. 3. When where the hydrolysis curve overlaps with the standard phosphate curve, all phosphate is released from ADP, indicating that hydrolysis is complete. At this level of ADP production, the amount of phosphatase is therefore adequate. However, as the level of ADP produced increases beyond the capacity for a given amount of phosphatase, hydrolysis may not be complete. At this point, coupling capacity is not adequate for the ADP production during a kinase reaction, and the hydrolysis curve diverges and falls below the phosphate standard curve. Therefore, the amount of phosphatase and the reaction time for a coupled kinase assay may be selected based on the predicted amount of ADP production and the corresponding coupling capacity, such that the coupling capacity is always larger than the predicted amounts of ADP production for the kinase reaction.

Another method of measuring the activity of a phosphatase for ADP includes measuring the coupling efficiency of the phosphatase, which varies depending upon the concentration of ATP used in the reaction. In particular, the phosphatase preferably has sufficiently greater activity with ADP than with ATP such that even under conditions in which the concentration of ATP is much greater than ADP, such as at the beginning of a kinase reaction, it nevertheless exhibits preferential activity with ADP. Under such conditions, there is the potential for the presence of high levels of ATP to competitively inhibit the activity of the phosphatase with ADP. The coupling efficiency of the phosphatase may be calculated and used to correct for any such inhibition caused by the presence of high levels of ATP.

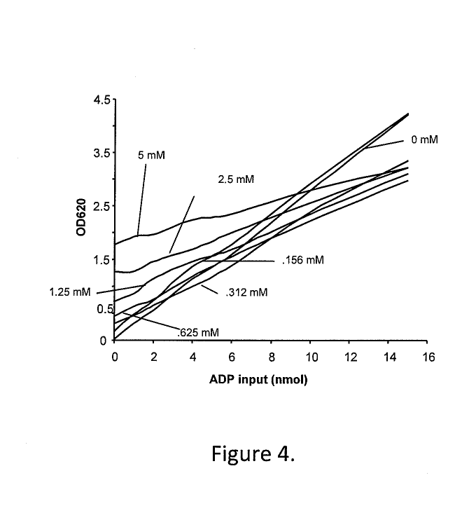

The coupling efficiency of a phosphatase may be determined by measuring the rates of ADP hydrolysis in the presence of ATP, and in the absence of ATP, under conditions which are otherwise the same. The coupling efficiency is the ratio of the ADP hydrolysis rate in the presence of ATP to the ADP hydrolysis rate in the absence of ATP. As exemplified in Example 3 (FIG. 4), the coupling efficiency in the presence of i mM of ATP, Cᵢ, is equal to the ratio of the slope of the corresponding ADP hydrolysis curve in the presence of i mM of ATP, Sᵢ, to the slope of the ADP hydrolysis curve in the absence of ATP, Sₒ. The coupling efficiency may be characterized by the following formula:

Cᵢ=Sᵢ/Sₒ (Eq. 1)

The coupling efficiency will vary depending upon the concentration of ATP. As such, the Cᵢ may be calculated at various ATP concentrations and the results may be plotted to give a curve of coupling efficiency versus ATP concentration. In this way, the coupling efficiency may be determined for any concentration of ATP, such as the beginning concentration of ATP used in a reaction. The coupling efficiency can be obtained once the conditions for a kinase reaction are determined and may then be used to adjust the final measured kinase activity depending upon the ATP concentration. For example, the final measured ADP production may be divided by the coupling efficiency (assuming that the ATP concentration throughout the reaction is approximately constant) in order to calculate the actual ADP production. The coupling efficiency allows for measuring the kinase activity even at high levels of ATP, such as at intracellular ATP concentration levels, about 1 mM, which may be most biologically relevant for some reactions.

After release of the phosphate from the ADP by the phosphatase, the free phosphate may be readily detected and/or measured by any means. Several methods are known for measuring free phosphate, any of which may be used. In some embodiments, the free phosphate may be detected and/or measured using a colorimetric assay. Examples of colorimetric assays for measurement of free phosphate which may be used in embodiments of the invention include the Malachite Green Phosphate Detection Kit available from R & D Systems, (Minneapolis, Minn.), PiColorlock™ Assay reagent available from Innova Biosciences, Ltd. (Cambridge, U.K.), and Phosphate Colorimetric Assay Kit available from BioVision (Mountain View, Calif.). In other embodiments, the free phosphate may be detected and/or measured by fluorescence detection. For example, free phosphate may be selectively detected by a fluorescent sensor as described in U.S. Pat. No. 7,521,250, the disclosure of which is hereby incorporated by reference. In another example, free phosphate may be detected using a recombinant E. coli phosphate-binding protein labeled with the fluorophore MDCC known as Phosphate Sensor and available from Invitrogen (Carlsbad, Calif.).

The Malachite Green Phosphate Detection Kit is one method that may be used to detect free phosphate and is based on the malachite green-molybdate binding reaction, and the kit itself, or the components or variations thereof, may be used in embodiments of the invention. The Malachite Green assay includes a first reagent, Malachite Green Reagent A, which includes ammonium molybdate and sulfuric acid, and a second reagent, Malachite Green Reagent B, which includes malachite green oxalate and polyvinyl alcohol. The Malachite Green assay further includes a phosphate standard, KH₂PO₄. The phosphate standard may be used to create a standard curve of absorbance at 620 nm for interpretation of sample assay results. The use of the assay includes incubating a sample with Malachite Green Reagent A for 10 minutes at room temperature, then adding Malachite Green Reagent B and incubating for 20 minutes at room temperature. The absorbance may then be read at 620 nm and compared to the phosphate standard curve to determine the amount of phosphate present in the sample.

The Malachite Green Phosphate Detection kit itself, or components or variations thereof, may therefore be used to detect levels of free phosphate released from ADP, according to embodiments of the invention. In such embodiments, known amounts of ATP, substrate, kinase, assay buffer, and phosphatase are combined to produce a sample for testing. In some embodiments, the sample may further include an additional component, such as a potential kinase inhibitor or promoter. At the completion of the reaction time, the resulting sample may be combined with Malachite Green Reagent A to stop the reaction, then incubated 10 minutes, and then combined with Malachite Green Reagent B and incubated an additional 20 minutes as described above. The absorbance may then be read at 620 nm using a spectrometer, and the reading may be correlated to a phosphate standard curve and/or a control (including all reaction components except the kinase enzyme), to determine the amount of free phosphate released by the phosphatase. This amount may be compared to the initial quantity of ATP and/or kinase present in the sample to determine the activity of the kinase and/or the affect of any additional components upon the kinase activity.

Embodiments of the invention include assays, kits and methods for detecting and measuring kinase activity. In some embodiments, the assay includes one or more of the following components: ATP; a phosphatase; a buffer; and a control kinase. The assay may optionally include free phosphate detection reagents, such as a first reagent comprising malachite green and a second reagent comprising molybdate.

In some embodiments, the kit may include a nucleotide phosphatase with preferential activity with ADP versus ATP and a free phosphate detection assay. In other embodiments, the kit may include ATP and a phosphatase with preferential activity with ADP versus ATP. In still other embodiments, the kit may include a phosphatase with preferential activity with ADP versus ATP, a free phosphate detection assay, and ATP. The kit may further include an assay buffer, ADP and/or a phosphate standard. The phosphate detection assay may be a Malachite Green detection assay. The ATP and/or ADP may be supplied in the assay buffer. The phosphatase may also be supplied in the assay buffer.

In one embodiment, the kinase assay kit includes an assay buffer, CD39L2 from either recombinant human or mouse origin, ATP, Malachite Green Reagent A, Malachite Green Reagent B, and a phosphate standard, such as KH₂PO₄. The kit may further include a kinase to be used as a positive control for the various components of the kit. The phosphatase, ATP and/or the control kinase may be provided in the assay buffer.

A kinase may be provided as a control in the kit, or may be supplied by the user of the kit. The control kinase serves to assure proper functioning of the assay. The assay may be performed using the control kinase and the results may be compared to known expected results for the kinase. If the results are within the expected range, the assay can be considered to be functioning properly. In this way, when the assay is performed using a kinase of interest, the results may be considered reliable. A control kinase provided in a kit is preferably stable over time and has a known activity, and the data regarding the control kinase activity and expected results may be provided with the kit. Examples of kinases which may be provided in kits to serve as controls include any stable kinase having a high specific activity and an available substrate. Examples include S. cerevisiae hexokinase and Mammanlian protein kinase A (PKA). S. cerevisiae hexokinase has a very high specific activity and uses glucose as the acceptor. Mammalian protein kinase A (PKA) has a broad array of substrates, including synthesized peptide.

The use of the kit may include first creating a free phosphate standard curve. The phosphate standard curve may be created using serial dilution of the phosphate standard in the assay buffer, and measuring the level of free phosphate using the phosphate detection assay. For example, each dilution may be combined with Malachite Green Reagent A and then with Malachite Green Reagent B as described and the absorbance may be read at 620 nm. The resulting measurements may be used to create a phosphate standard curve which may be used to calculate a conversion factor for the amount of phosphate corresponding to the measured OD for experimental reactions.

In order to correlate assay results to levels of free phosphate, and thereby to kinase activity, a standard curve such as a phosphate standard curve may be produced. In embodiments in which a Malachite Green assay is used to measure free phosphate, and in embodiments using other free phosphate detection methods as well, the phosphate standard curve may be made using serial dilutions, such as 2-fold serial dilutions, of a phosphate solution such as the phosphate standard, in the assay buffer. For example, the serial dilutions may be as in Table 1, below.

TABLE 1

| | | Phosphate input |
|---|---|---|
| Well | Phosphate concentration (μm) | (nmole) |
| 1 | 50 | 5,000 |
| 2 | 25 | 2,500 |
| 3 | 12.5 | 1,250 |
| 4 | 6.25 | 625 |
| 5 | 3.13 | 313 |
| 6 | 1.56 | 156 |
| 7 | 0.78 | 78 |
| 8 | 0 | 0 |

When the Malachite Green assay is used, for example, the serial phosphate dilutions may be added to a clear 96-well plate and may be performed in triplicate. The Malachite Green Reagent A is first added to each well, followed by the Malachite Green Reagent B. After 20 minutes, the optical density is read at 620 nm (OD620) for each well using a microplate reader or spectrophotometer. The average reading for each dilution may be obtained. The phosphate input may be plotted against the results, or the average of the results for each dilution, to create a standard curve, such as by using linear regression or a computer generated four parameter logistic (4-PL) curve fit. A similar curve may be obtained using other free phosphate detection methods or other phosphate sources. The slope of the linear regression line may be used as the conversion factor to determine how much phosphate corresponds to each absorbance unit. This conversion factor may then be used to calculate the amount of free phosphate from the measured absorbance for each reaction.

In some embodiments, a single buffer is used which is the assay buffer. The assay buffer should allow the kinase, and preferably also the phosphatase, to function normally. In some embodiments, the assay buffer may contain about 10 mM divalent cations and may have a pH of about 7.0 to about 8.0. In some embodiments, the assay buffer may comprise 25 mM Tris, 150 mM NaCl, 5 mM MgCl₂, and 5 mM CaCl₂ at pH 7.5. In other embodiments, two or more buffers may be used. The first buffer may be an assay buffer and the second buffer may be a phosphatase buffer. For example, the phosphatase may be divalent cation dependent, and therefore the phosphatase buffer should include the divalent cation, while the assay buffer might not have this component.

In some embodiments, the buffer and other reagents have low levels of divalent cations such as calcium, magnesium and manganese. In some embodiments, the phosphatase may be active in the assay buffer used with or provided with the assay. In such embodiments, the phosphatase may be combined with the ATP, substrate, and kinase, in the same buffer. In other embodiments, the phosphatase is not active in the assay buffer used with or provided with the assay. In such embodiments, the ATP, substrate, and kinase may first be combined in a first buffer which is the assay buffer. A second buffer which is the phosphatase buffer may then be added. The phosphatase may be added with the phosphatase buffer or may be added separately, after addition of a sufficient amount of the phosphatase buffer. The phosphatase buffer may be stronger than the kinase assay buffer, such that the conditions provided by the phosphatase buffer will overwhelm those provided by the assay buffer, with the resulting mixture being more similar to the phosphatase buffer and therefore being favorable for phosphatase activity.

The substrate used in embodiments of the invention may be any substrate which is phosphorylated by a kinase. Such substrates include sugars, proteins, lipids, or any conjugates of these. Any substrate which is acted upon by a kinase of interested may be used in the assay, with the exception of the nucleotides ADP, GDP, UDP, TDP, CDP, AMP, GMP, TMP and CMP. As such, the substrate may be any substrate except a nucleotide having one or two phosphates at the 5′ position.

Sources of stored ATP may have the potential to degrade over time, to generate ADP. The presence of ADP in the ATP used in embodiments of the invention may lead to inaccurate measurements of kinase activity, as this ADP would release free phosphate which would be measured but would not represent kinase activity. In order to compensate for any background measurement not due to kinase activity, including ADP present in the ATP, a control may be run including all components of the assay except the kinase. That is, the control could include ATP, the substrate, the phosphatase selective for ADP, the assay buffer, and the free phosphate detector. Free phosphate (or OD) would then be measured, and this measurement could be deducted from the subsequent tests using the same components and the kinase. However, it is preferable that the ATP be as stable over time and pure as possible, so that the phosphate measurement provides a measurement of kinase activity with less adjustment required for background signal.

Because of their key roles in diverse biological activities, kinases may be ideal targets for drug therapies, to promote, inhibit, or block their activities. Embodiments of the invention may be used in the development of such drug therapies and can be used to identify and evaluate the effectiveness of agents at altering the activity of a kinase, such as promoting, inhibiting or blocking the activity of the kinase. For example, embodiments of the invention may be used to screen possible agents for an effect upon kinase activity. In some embodiments of the invention effectiveness of an proposed agent, at inhibiting or blocking the activity of a kinase is evaluated. In still other embodiments, the assay may be used for determining the dose response of kinase to an agent. When embodiments of the invention are used for evaluating a potential therapeutic agent, the reactions may be performed as described above with the addition of the proposed therapeutic agent to the kinase reaction. The kinetics of the reaction including the proposed therapeutic agent may be compared to the same kinase reaction in the absence of the therapeutic agent (as a control) to determine the effect of the agent. For example, the OD, ODₘₐₓ, free phosphate, specific activity, Vₘₐₓ, or Kₘ may be compared with and without the agent to determine what effect the agent had upon the kinase reaction, and to obtain IC50 (half maximal inhibitory concentration) values if the agent is inhibitory.

Substances which may be assayed as test substances include potential drugs or therapeutic agents include, for example, small molecule kinase inhibitors and recombinant monoclonal antibodies.

### Experimental

In the following examples, hexokinase of Saccharomyces cerevisiae, ATP, ADP, glucose, mannose, galactose, N-acetylglucosamine and methyl α-D-mannopyranoside were obtained from Sigma-Aldrich (St. Louis, Mo.). Recombinant human CD39L2 (ENTPD6), mouse CD39L2, recombinant adenosine 5′-phosphosulfate kinase (APSK) of Penicillium chrysogenum, extracellular signal-regulated kinase (ERK1), and Malachite Green Phosphate Detection kit were obtained from R&D Systems (Minneapolis, Minn.). Recombinant human CD39L2 (ENTPD6) had a specific activity >20,000 pmol/min/μg and recombinant mouse CD39L2 had a specific activity >100,000 pmol/min/μg. The amount of phosphatase used in each reaction was based on the predicted ADP production in the reaction. For a typical kinase reaction that releases up to 20 nmol ADP (0.4 mM), 0.2 μg human CD39L2 was used for coupling. When mouse CD 39L2 is used, 40 ng of the mouse CD39L2 can be substituted for 0.2 μg human CD39L2 for the same reaction, as the mouse enzyme is more active and therefore has a higher coupling capacity

The kinase reactions were carried out in 50 μL of kinase assay buffer including 25 mM Tris, 150 mM NaCl, 5 mM MgCl₂, and 5 mM CaCl₂ at pH 7.5 in a 96 well plate at room temperature. Each reaction was stopped with 30 μL of Malachite Reagent A and 100 μL of water. The color was developed with 30 μL Malachite Reagent B. The plate was read at 620 nm with a multi-well plate reader. If the predicted phosphate content in the reaction was above 4,000 pmol, the reaction was diluted before phosphate detection.

To determine kinetic parameters of the kinase, multiple reactions with varying amounts of the enzyme or substrate were performed simultaneously in the presence of fixed amounts of all other components. For determining the Kₘ and Vₘₐₓ values, the results were plotted against substrate concentrations and fitted into the Michaelis-Menten equation using KaleidaGraph 4.

### Example 1

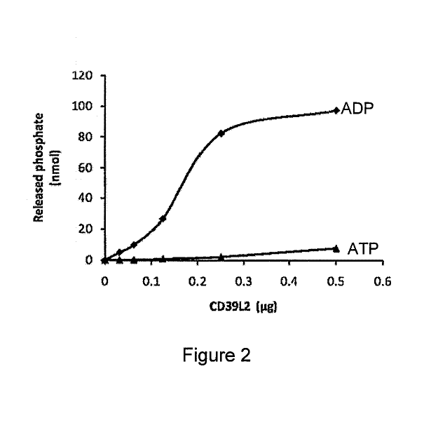

In this experiment, the relative activity of rhCD39L2 on ADP and ATP was determined. In a first series of reactions, 100 nmol of 2 mM ATP was combined with increasing amounts of rhCD39L2 (0.03125 μg, 0.0625 μg, 0.125 μg, 0.25 μg, 0.5 μg) for 20 minutes. Likewise in a second series of reactions, 100 nmol of 2 mM ADP was combined with the same increasing amounts of rhCD39L2 for 20 minutes. The reactions were stopped after 20 minutes and free phosphate was measured using the Malachite Green assay as described above. The results are shown in FIG. 2.

As can be seen in FIG. 2, although the phosphatase was able to release a small amount of phosphate from ATP, it released much greater amounts from ADP. Identical amounts of rhCD39L2 released much more phosphate from ADP than from ATP. For example, 0.25 μg of the phosphatase was able to release about 80 nmol phosphate from ADP, but it released less than 3 nmole phosphate from ATP. At the rising phases of the ADP hydrolysis curve, rhCD39L2 released 35 fold more phosphate from ADP than from ATP. Because ATP has two hydrolyzable phosphate residues, this suggests that rhCD39L2 is about 70 fold more active on ADP. Based on this experiment, 0.2 m of rhCD39L2 was chosen for coupling for the kinase reactions in Examples 2-7 below in order to maximize the hydrolysis activity on ADP while minimizing the activity on ATP.

### Example 2

In order to provide a measurement of kinase activity, the phosphatase needs to be able to completely hydrolyze all ADP generated by the kinase reaction. In this example, the coupling capacity was determined by performing three series of reactions combining 0.2 μg rhCD39L2 with increasing amounts of ADP (3.125, 6.25, 12.5, 25, 50, and 100 nmols), with each series of reactions allowed to run for various lengths of time 10, 20 or 40 minutes. The reactions were stopped and free phosphate was measured using the Malachite Green assay as described above. The results are shown in FIG. 3, which also includes a phosphate standard curve as a dashed line, shown for comparison. The coupling capacity of 0.2 μg rhCD39L2 was found to be 10, 20, and 100 nmole of ADP for 10, 20 and 40 minute reactions, respectively. Based on these results, examples 3-5 and 7 were performed using 0.2 μg rhCD39L2 for coupling a 20 minute kinase reaction, because this amount of phosphatase would be capable of reacting with up to 20 nmol ADP in this amount of time. Because the relatively large coupling capacity (20 nmol) would be well above the expected amount of ADP production in those examples, a 20 minute reaction time with 0.2 μg of CD39L2 was chosen out of convenience. In cases where ADP production is lower, a shorter reaction time or less CD39L2 can be utilized. In example 6, a 10 minute reaction was used for the same amount of enzyme, because the amount of ADP generated was less than 2 nmol, and the amount of enzyme was therefore able to couple the APSK reaction in 10 minutes.

### Example 3

In some situations, the concentration of ATP will be much higher than that of ADP, such as at the start of a reaction. Under such conditions, even though the phosphatase may be much less active on ATP than ADP, the presence of a high relative concentration of ATP could competitively inhibit the activity of the phosphatase with ADP by binding the enzyme. As a result, the activity of the kinase could be underestimated. However, this experiment shows that it is possible to compensate for this effect using the coupling efficiency of the phosphatase.

This experiment measured the coupling efficiency of CD39L2, which was the ratio of the rate of ADP hydrolysis in the presence of ATP to the rate of ADP hydrolysis in the absence of ATP, under conditions which were otherwise identical. In a series of reactions, increasing amounts of ADP (0 to 15 nmol, within the coupling capacity) were hydrolyzed with 0.2 μg of rhCD39L2 in the presence of various concentrations of ATP (0, 0.156, 0.312, 0.625, 1.25, 2.5, or 5 mM). The reactions were stopped after 20 minutes and the Malachite Green assay was performed and OD readings were taken at 620 nm. The resulting data was plotted as ADP hydrolysis curves for each concentration of ATP as shown in FIG. 4.

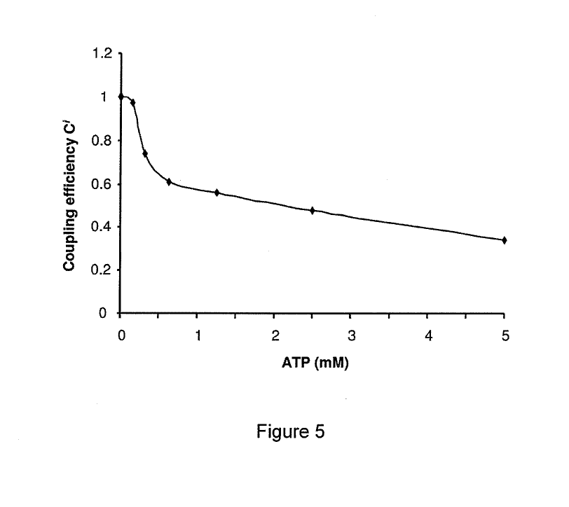

Coupling efficiency in the presence of i mM of ATP (Cᵢ) was determined from the ratio of the slope of the ADP hydrolysis curve in the presence of i mM ATP (Sᵢ) to the slope of the ADP hydrolysis curve in the absence of ATP (S₀) according to Equation 1. For each concentration of ATP, Cᵢ was calculated. The results are shown in Table 1 below and were then plotted against ATP concentration, as shown in FIG. 5. As can be seen, Ci decreased rapidly between 0.125 mM and 0.5 mM of ATP, then decreased gradually beyond 0.5 mM of ATP and finally dropped to 0.34 at 5 mM of ATP. The coupling efficiency may then be used to correct the data obtained for any competitive inhibition that might be caused by the ATP, especially at high concentrations of ATP.

TABLE 2

| ATP | Slope of ADP | Linear Correlation | Coupling |
|---|---|---|---|
| (mM) | Hydrolysis Curve (Sᵢ) | Coefficient (R²) | Efficiency (Cᵢ) |
| 0 | 0.280 | 0.999 | 1.00 |
| 0.156 | 0.271 | 0.999 | 0.97 |
| 0.312 | 0.207 | 0.997 | 0.74 |
| 0.625 | 0.170 | 0.997 | 0.61 |
| 1.25 | 0.157 | 0.997 | 0.56 |
| 2.50 | 0.134 | 0.997 | 0.48 |
| 5.0 | 0.096 | 0.996 | 0.34 |

### Example 4

In this experiment, the specific activity of Saccharomyces cerevisiae hexokinase was measured. Because hexokinase catalyzes the conversion of glucose to glucose-6-phosphate, the first committed step of glucose metabolism, it is an essential enzyme to all organisms that use glucose as an energy source. Traditionally, the activity of hexokinase has been measured by using hexokinase to generate glucose-6-phosphate, which then acts as a substrate for glucose-6-phosphate dehydrogenase to generate nicotinamide adenine dinucleotide phosphate (NADPH), which can then be measured by using a spectrometer to read absorbance at 340 nm. In contrast, in this experiment, the activity of Saccharomyces cerevisiae hexokinase was measured more easily and more directly by measuring the free phosphate released from the product ADP by coupling phosphatase CD39L2.

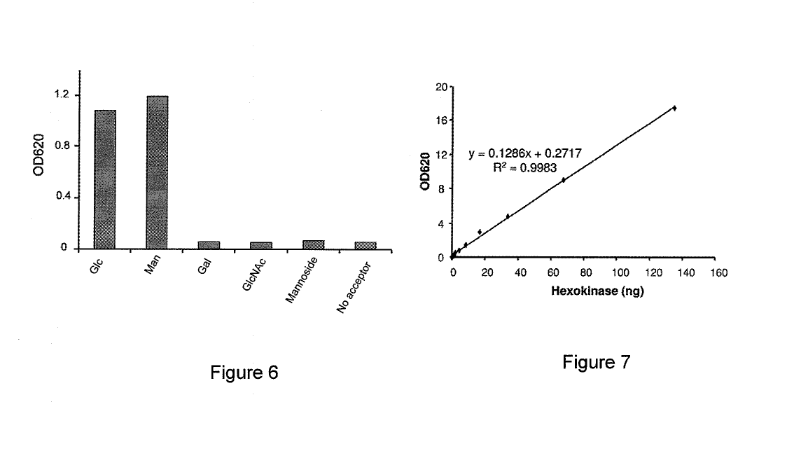

First, a series of reactions were performed combining 0.1 μg Saccharomyces cerevisiae hexokinase with 2 mM of the following potential acceptor substrates: glucose, mannose, galactose, N-acetylglucosamine, and methyl α-D-mannopyranoside. Each reaction was performed in the presence of 0.1 mM ATP and 0.2 μg recombinant human CD39L2 (rhCD39L2). The reactions were stopped after 20 minutes, the Malachite Green assay was used as described above, and OD readings were taken at 620 nm. The results are shown in FIG. 6. As shown in the figure, the hexokinase only had significant activity when glucose and mannose were the substrates.

Next, a series of reactions were performed by mixing increasing amounts of Saccharomyces cerevisiae hexokinase with 2 mM of ATP, 2 mM glucose and 0.2 μg rhCD39L2. The amount of hexokinase used was 0 to 140 ng. The reactions were stopped after 20 minutes, the Malachite Green assay was performed as described above, and the OD was measured at 620 nm. The results are shown in FIG. 7, wherein the OD reading (or calculated reading if diluted before the measurement) is plotted against the amounts of hexokinase. This plot was then used to determine the specific activity of Saccharomyces cerevisiae hexokinase. The specific activity was calculated by first determining the slope of the curve (0.1286 OD/ng). The slope was then multiplied by a conversion factor (4081 pmol/OD) and divided by the reaction time (20 minutes) to give an uncorrected specific activity of 26.24 pmol/ng/min. (0.1286 OD/ng×4081 pmol/OD/20 min=26.24 pmol/ng/min) The conversion factor had been determined in a separate experiment with inorganic phosphate as described above. The specific activity was then corrected to account for coupling efficiency. The coupling efficiency of rhCD39L2 in 2 mM ATP had been determined to be 0.51 based on data of Example 3. The uncorrected specific activity of the hexokinase was then adjusted by dividing by the coupling efficiency, and the final specific activity was calculated to be 51.5 pmol/min/ng. (26.24 pmol/ng/min/0.51=51.5 pmol/min/ng)

### Example 5

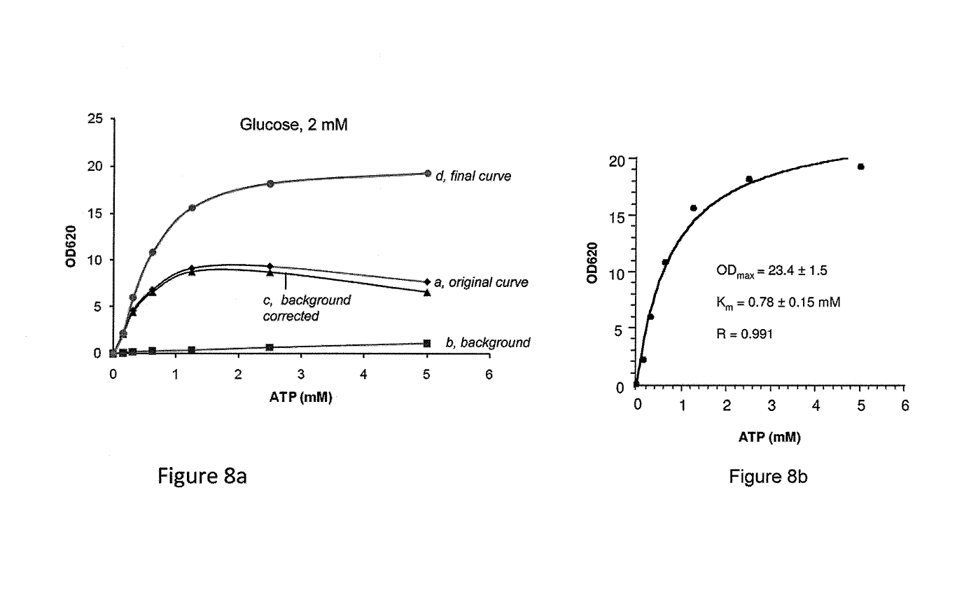

A series of reactions was performed using 67 ng Saccharomyces cerevisiae hexokinase, glucose (10 mM), 0.2 μg CD39L2, and various quantities of ATP (0, 0.156, 0.312, 0.625, 1.25, 2.5, and 5 mM). The reactions were stopped after 20 minutes, assayed with the Malachite Green assay as described above and OD readings were measured at 620 mm. The results are shown as line a in FIG. 8a. A second series of reactions was performed under the same conditions, except in the absence of Saccharomyces cerevisiae hexokinase, to be used as a control for background correction. The reactions were likewise stopped and assayed with Malachite Green and OD readings were measured at 620 nm, and the results are shown as line b of FIG. 8a. Line b was subtracted from line a to produce line c, which represents the activity of Saccharomyces cerevisiae hexokinase after adjusting for background noise. Finally, curve c was adjusted by the coupling efficiency to obtain curve d. Curve d represents the true ADP formed by the kinase reaction, obtained after background correction and coupling efficiency correction. In this example, the experiment was performed at high levels of ATP, such that correction of the data by the coupling efficiency was needed to avoid underestimation of the actual kinase activity.

Curve d was then fitted into the Michaelis-Menten equation to obtain the curve shown in FIG. 8b. From this curve, Kₘ was found to be of 0.79+/−0.15 using KaleidaGraph 4 software. Vₘₐₓ can be determined from the ODₘₐₓ by using the conversion factor, previously determined to be 4081 pmol/OD. Using ODₘₐₓ (23.4), the conversion factor (4081 pmol/OD), the reaction time (20 minutes), and the enzyme amount (67 ng), Vₘₐₓ was calculated to be to be 71 pmol/min/ng. (23.4 OD×4081 pmol/OD/20 mins/67 ng=71 pmol/min/ng)

### Example 6

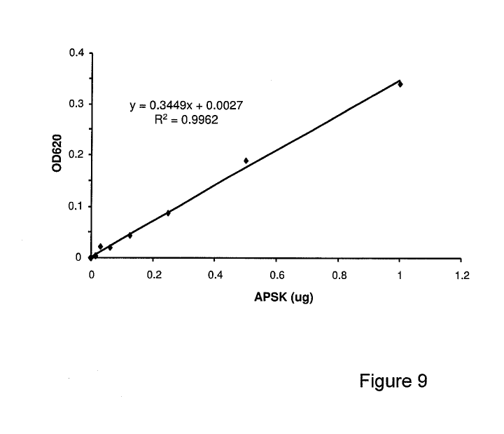

A series of reactions were performed as in Example 4, but recombinant adenosine 5′-phosphosulfate kinase (APSK) of Penicillium chrysogenum (EC2.7.1.25), was used as the kinase. Various amounts of APSK (0 to 1 μg) were mixed with 0.2 mM ATP, 0.1 mM of adenosine 5′-phosphosulfate (APS) and 0.2 μg rhCD39L2. The reactions were stopped after 10 minutes, the Malachite Green assay was performed as described above, and the OD was measured at 620 nm. The results are shown in FIG. 9, wherein the OD reading is plotted against APSK. This plot was then used to determine the specific activity of APSK. The specific activity was calculated by first determining the slope of the curve (0.3449 OD/μg). The slope was then multiplied by a conversion factor (4081 pmol/OD) and divided by the reaction time (10 minutes) (0.3449 OD/μg×4081 pmol/OD/10 minutes=140.75 pmol/min/μg). The coupling efficiency of rhCD39L2 in 0.2 mM ATP had been determined to be 0.9 based on the data in Example 3. The specific activity of the APSK was then adjusted for the coupling efficiency and was calculated to be 156.39 pmol/min/μg (140.75 pmol/min/ug/0.9=156.39 pmol/min4 μg).

### Example 7

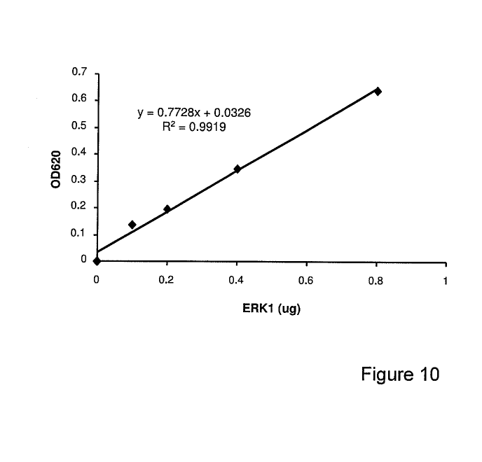

A series of reactions were performed with extracellular signal-regulated kinase 1 (ERK1) as the kinase. Various amounts of ERK1 (0, 0.1, 0.2, 0.4 and 0.8 μg) were combined with 4 mM ATP, 4 mM MBP peptide acceptor substrate, and 0.2 μg CD39L2. The MBP peptide had a sequence of CVTPRTPPPSQ-OH. The reactions were stopped after 20 minutes, the Malachite Green assay was performed as described above, and the OD was measured at 620 nm. The results are shown in FIG. 10, wherein the OD reading is plotted against the quantity of ERK1. This plot was then used to determine the specific activity of ERK1. The specific activity was calculated by first determining the slope of the curve (0.7728 OD/μg), which was then multiplied by a conversion factor (4081 pmol/OD) and divided by the reaction time (20 minutes) (0.7728 OD/μg×4081 pmol/OD/20 min=157.69 pmol/min/μg). The coupling efficiency of rhCD39L2 in 4 mM ATP was predicted to be 0.4 based on the data of Example 3 (FIG. 5). The specific activity of the ERK1 was then adjusted for the coupling efficiency and was calculated to be 394.22 pmol/min/μg. (157.69 pmol/min/μg/0.4=394.22 pmol/min/μg)

### Example 8

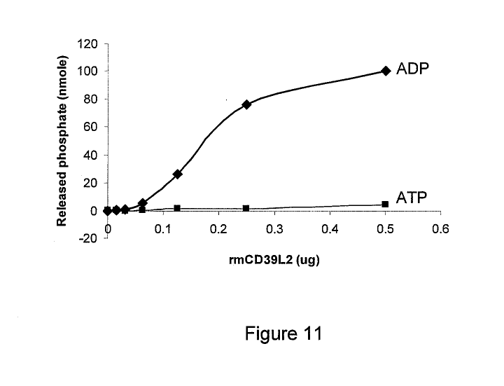

The relative activity of recombinant mouse CD 39L2 (rmCD39L2) on ADP and ATP was determined in a series of reactions similar to Example 1. 100 nmol of 2 mM ATP was combined with increasing amounts of rmCD39L2 (0.03125 μg, 0.0625 mg, 0.125 0.25 μg, 0.5 μg). Likewise in a second series of reactions, 100 nmol of 2 mM ADP was combined with the same increasing amounts of rmCD39L2. The reactions were stopped after 5 minutes and free phosphate was measured using the Malachite Green assay as described above. The results are shown in FIG. 11. As can be seen in the figure, rmCD39L2 shows a preference of about 30-40 times greater activity with ADP than with ATP when the phosphatase was present at 0.25 μg.

### Example 9

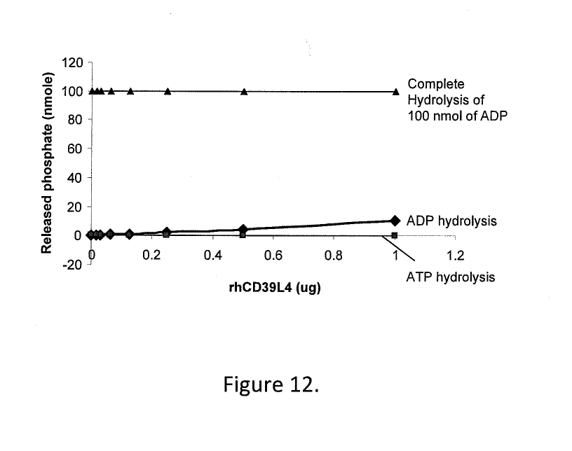

The experiment of Example 1 was repeated using recombinant human CD39L4 (rhCD39L4). 100 nmol of ATP (2 mM) was combined with increasing amounts of rhCD39L4 (0.03125 μg, 0.0625 μg, 0.125 μg, 0.25 μg, 0.5 μg, 1.0 μg). Likewise in a second series of reactions, 100 nmol of ADP (2 mM) was combined with the same increasing amounts of rhCD39L4 for 5 minutes. The reactions were stopped after 5 minutes and free phosphate was measured using the Malachite Green assay as described above. The results are shown in FIG. 12. As can be seen in the figure, rhCD39L4 was about 10 to 20 times more active with ADP than with ATP. As such, the necessary preference of the phosphatase for ADP as compared to ATP is present. Although rhCD39L4 had a lower level of activity than rhCD39L2, not reaching complete hydrolysis in the reactions shown, coupling reactions with rhCD39L4 may be performed by compensating for this lower activity by using greater amounts of the phosphatase.

In the foregoing detailed description, the invention has been described with reference to specific embodiments. However, it may be appreciated that various modifications and changes can be made without departing from the scope of the invention. Thus, some of the features of preferred embodiments described herein are not necessarily included in preferred embodiments of the invention which are intended for alternative uses.

1. An assay for detecting activity of a test kinase comprising:
a nucleotide phosphatase, wherein the phosphatase is at least ten times more active on ADP than on ATP;
a free phosphate detector; and
a control kinase.

2. The assay of claim 1 wherein the phosphatase is an ectonucleoside triphosphate diphosphohydrolase.

3. The assay of claim 2 wherein the phosphatase is ectonucleoside triphosphate diphosphohydrolase 6.

4. The assay of claim 1 wherein the phosphatase is at least about 30 times more active on ADP than on ATP.

5. The assay of claim 1 wherein the phosphatase is at least about 70 times more active on ADP than on ATP.

6. The assay of claim 1 further comprising a source of free phosphate for use as a phosphate standard.

7. The assay of claim 1 wherein the free phosphate detector comprises a colorimetric assay.

8. The assay of claim 7 wherein the free phosphate detector comprises a first reagent and a second reagent, wherein the first reagent comprises ammonium molybdate and the second reagent comprises malachite green oxalate.

9. The assay of claim 1 wherein the control kinase comprises a Saccharomyces cerevisiae hexokinase.

10. The assay of claim 1 further comprising ATP.

11. An assay for detecting kinase activity comprising:
ATP;
a nucleotide phosphatase, wherein the phosphatase is at least ten times more active on ADP than on ATP; and
a free phosphate detector.

12. The assay of claim 11 wherein the phosphatase is an ectonucleoside triphosphate diphosphohydrolase.

13. The assay of claim 11 wherein the free phosphate detector comprises a colorimetric assay.

14. A method of detecting kinase activity comprising conducting a first reaction comprising:
combining a kinase, a substrate of the kinase, and a known quantity of ATP under conditions to produce ADP;
combining the produced ADP with a phosphatase specific for ADP;
measuring free phosphate.

15. The method of claim 14 further comprising calculating kinase activity using the measured amount of free phosphate.

16. The method of claim 14 further comprising adjusting the measured free phosphate according to a coupling efficiency based on the quantity of ATP used in the reaction.

17. The method of claim 14 wherein measuring the free phosphate comprises measuring optical density.

18. The method of claim 14 further comprising conducting a second reaction comprising combining the substrate of the kinase and the known quantity of ATP with the phosphatase and measuring free phosphate, wherein the second reaction provides a background control.

19. The method of claim 18 further comprising reducing the measured phosphate of the first reaction by the measured phosphate of the second reaction to calculate the amount of phosphate produced by the kinase.

20. The method of claim 14 wherein the phosphatase is an ectonucleoside triphosphate diphosphohydrolase.

21. The method of claim 20 wherein the phosphatase is ectonucleoside triphosphate diphosphohydrolase 6.

22. The method of claim 14 wherein the phosphatase is at least about 10 times more active on ADP than on ATP.

23. The method of claim 14 wherein the phosphatase is at least about 30 times more active on ADP than on ATP.

24. The method of claim 14 wherein measuring free phosphate comprises applying a colorimetric free phosphate detection assay to the first reaction.

25. The assay of claim 24 wherein the free phosphate detection assay comprises a first reagent and a second reagent, wherein the first reagent comprises ammonium molybdate and the second reagent comprises malachite green oxalate.
